(19) United States
(12) Patent Application Publication
(10) Pub. No.: US 2024/0065844 A1
(43) Pub. Date: Feb. 29, 2024

(54) FIXATION ASSEMBLY FOR SECURING MEDICAL IMPLANT IN PATIENT

(71) Applicant: FORMAE, INC., Paoli, PA (US)

(72) Inventor: Kevin A Mansmann, Paoli, PA (US)

(73) Assignee: FORMAE, INC., Paoli, PA (US)

(21) Appl. No.: 18/258,078

(22) PCT Filed: Dec. 15, 2021
(86) PCT No.: PCT/US2021/063485

§ 371 Date: Jun. 16, 2023

Related U.S. Application Data
(60) Provisional application No. 63/126,256, filed on Dec. 16, 2020.

(51) Int. Cl. A61F 2/30

(52) U.S. Cl. CPC A61F 2/30749; A61F 2002/30069; A61F 2002/30331; A61F 2002/30621; A61F 2002/3085

(57) ABSTRACT

A device configured for use as a medical implant is disclosed. The device includes an anchor body including a first end configured to retain an elastic articulating component and a second end including a collar defining a receptacle configured to receive a portion of a fixation element. The fixation element can include a head portion and a threaded portion. The head portion is configured to be inserted and retained within the receptacle and the threaded portion is configured to retain the fixation element within a patient recipient site.

### INCORPORATION BY REFERENCE

This application claims the benefit of U.S. Provisional Patent Application 63/126,256, filed on Dec. 16, 2020, which is incorporated herein by reference as if fully set forth herein.

### FIELD OF INVENTION

The present invention relates to medical implants, and is more particularly related to anchoring systems for medical implants.

### BACKGROUND

As explained in U.S. Patent Publication No. 2018/0289493, which is commonly owned by the present Applicant (Formae, Inc.), the entire contents of which is incorporated by reference as if fully set forth herein, medical implants including a hollow anchor having a cavity-like recess or receiving area with a raised or surrounding sidewall, rim, ridge or lip, for attaching a cartilage-like flexible material to a bone are known. PCT Application PCT/US2020/063539, which is also owned by the present Applicant (Formae, Inc.), describes another known type of implant, and is also incorporated by reference as if fully set forth herein.

Difficulty in anchoring medical implants to bone has been encountered. Accordingly, it would be desirable to provide a more effective and reliable way to secure an implant to a patient.

### SUMMARY

A device configured for use as a medical implant that includes a fixation element is disclosed herein. The device includes an anchor body having a first end defining a cavity, and an elastic articulating component retained within the cavity. A second end of the anchor body includes a fixation element extending cantilevered from the anchor body.

The second end of the anchor body can define a receptacle, and the fixation element can be secured within the receptacle. The fixation element includes a threaded portion that is configured to retain the implant with a patient recipient site. A first width (W1) of the cavity is larger than a second width (W2) of the receptacle, in one aspect.

The fixation element can be formed separately from the anchor body, and the fixation element can further comprise a head portion dimensioned to be retained within the receptacle. The head portion can include a drive interface configured to be engaged by a tool. The anchor body can be configured to be impacted in a downward direction to secure the anchor body onto the head portion, and the head portion can engage against a base of the anchor body in an installed state. The fixation element is formed integrally with the anchor body, in one aspect.

The anchor body can include a receptacle having a first tapered profile that is a female Morse taper, and the fixation element can include a head portion having a second tapered profile that is a male Morse taper. The female Morse taper of the receptacle and the male Morse taper of the head portion are configured to secure the anchor body with the fixation element via a cold weld or bond.

An interface region (IR) can be defined between the first and second tapered profiles, and the interface region (IR) can be shorter than an axial length (Ls) of a threaded portion of the fixation element. The interface region (IR) can be at least 50% of an axial depth of the receptacle.

The anchor body can further include a sidewall partially defining the cavity, and a collar defining a receptacle, wherein the sidewall and the collar each have an outer surface layer having a porosity configured to promote bone ingrowth.

The anchor body can define a base that partially defines the cavity on a first surface and defines an attachment surface for the fixation element on a second, opposite surface.

The anchor body can define a base that partially defines the cavity on a first surface and defines an abutment surface for a head portion of the fixation element on a second, opposite surface.

In another aspect, a device configured for use as a medical implant is disclosed that includes an anchor body having a first end having a sidewall partially defining a cavity, and at least one attachment element configured to retain an elastic articulating component. A second end of the anchor body includes a collar defining a receptacle having a first tapered profile that is configured to receive a portion of a fixation element. The device also includes a fixation element including a head portion and a threaded portion. The head portion has a second tapered profile, and the head portion is configured to be inserted and retained within the receptacle via engagement between the first and second tapered profiles. The threaded portion is configured to retain the device with a patient recipient site.

The anchor body can include a base that partially defines the cavity on a first surface, and the base defines a second, opposite surface directly facing the head portion of the fixation element. The base can include the at least one attachment element configured to retain the elastic articulating component. A first width (W1) of the cavity can be larger than a second width (W2) of the receptacle. The sidewall and the collar can both include an outer layer having a porosity configured to promote bone ingrowth.

In another aspect, a device configured for use as a medical implant is also disclosed. The device includes an anchor body having a first end having a sidewall partially defining a cavity, and at least one attachment element configured to retain an elastic articulating component. A second end of the anchor body includes a collar that is integrally formed with the sidewall and defines a receptacle having a first tapered profile that is configured to receive a portion of a fixation element. A fixation element can include a head portion and a threaded portion. The head portion has a second tapered profile, and the head portion is configured to be inserted and retained within the receptacle via engagement between the first and second tapered profiles. The threaded portion is configured to retain the device with a patient recipient site.

The sidewall and the collar can have a constant outer diameter, and both include an outer layer having a porosity configured to promote bone ingrowth.

The anchor body includes a base that partially defines the cavity on a first surface, and the base defines a second, opposite surface directly facing the head portion of the fixation element.

Additional aspects and embodiments are described herein.

### BRIEF DESCRIPTION OF THE DRAWINGS

The foregoing Summary and the following detailed description will be better understood when read in conjunction with the appended drawings, which illustrate a preferred embodiment of the invention. In the drawings:

FIG. 1A illustrates a perspective view of an implant according to one aspect.

FIG. 1B illustrates another perspective view of the implant of FIG. 1A.

FIG. 1C illustrates a fixation element configured to be used with the implant of FIGS. 1A and 1B.

FIG. 2A illustrates a side view of the fixation element and the implant prior to attachment with each other.

FIG. 2B illustrates another side view of the fixation element and the implant prior to attachment with each other.

FIG. 3 is a cross sectional view of the fixation element and the implant in an engaged or attached state.

FIG. 4A is a side view of another aspect of an implant including an integrally formed threaded portion.

FIG. 4B is a perspective view of the implant of FIG. 4A.

FIG. 5 is a cross sectional view of another embodiment of the implant.

### DETAILED DESCRIPTION OF THE PREFERRED EMBODIMENTS

The description provided herein is to enable those skilled in the art to make and use the described embodiments set forth. Various modifications, equivalents, variations, combinations, and alternatives, however, will remain readily apparent to those skilled in the art. Any and all such modifications, variations, equivalents, combinations, and alternatives are intended to fall within the spirit and scope of the present invention defined by claims.

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

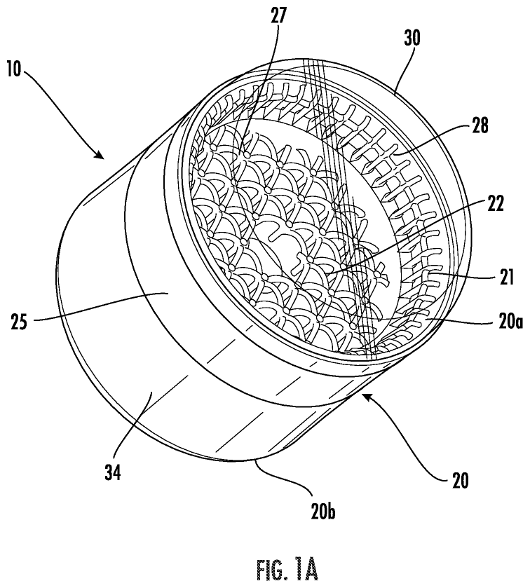

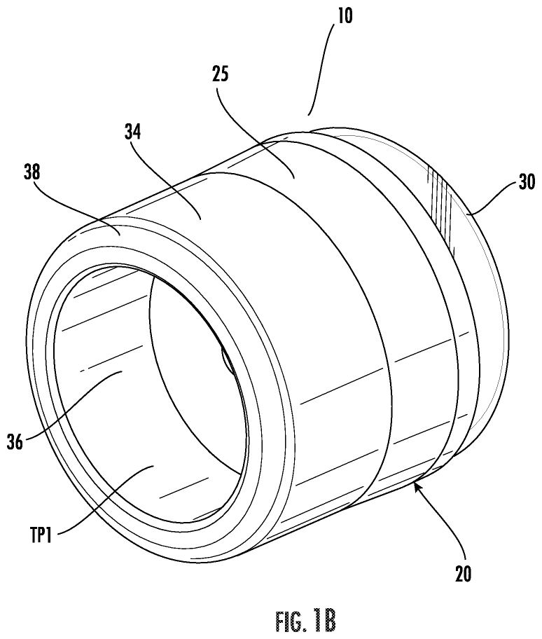

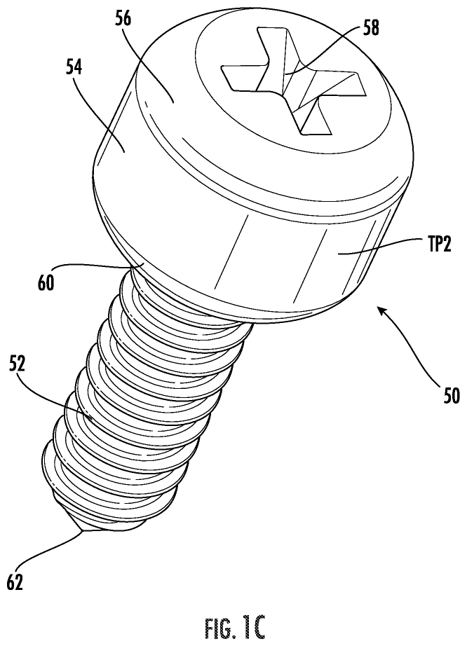

As shown in FIGS. 1A and 1B, an implant 10 is disclosed herein that is configured to be installed within a patient's bone. In one aspect, the implant 10 is configured to be implanted into a patient's femur, and more specifically on an end of the femur that engages the tibia. One of ordinary skill in the art would recognize that the implant 10 could be used on any other bone. Additional details of the implant 10 are disclosed in PCT Application PCT/US2020/063539, which is incorporated by reference as if fully set forth herein. As shown in FIGS. 1A, and 1B, the implant 10 generally includes an anchor body 20 and an elastic articulating component 30. The anchor body 20 can include a first end 20a defining a cavity 21, and the elastic articulating component 30 is retained within the cavity 21. A second end 20b of the anchor body 20, which is opposite from the first end 20a, can include a fixation element, such as fixation elements 50, 150, 250. In one aspect, the fixation elements 50, 150, 250 can extend cantilevered from the anchor body 20. Within the anchor body 20, at least one attachment element can be provided to secure the elastic articulating component 30 with the anchor body 20. Specifically, in one aspect, a first attachment element 27 can be provided along a base 22 of the anchor body 20 and a second, separate attachment element 28 can be provided around a rim on the first end 20a of the anchor body 20. In one aspect, the attachment elements 27, 28 can include a lattice structure. One of ordinary skill in the art would understand that various arrangements for attachment elements or other retention elements could be provided.

The elastic articulating component 30 can be formed from a hydrogel, and can include a polymeric molecular matrix that is configured to cohesively hold water molecules. The elastic articulating component 30 is configured to provide some degree of deformability such that the elastic articulating component 30 returns to a specific non-deformed shape after the application of loads or stresses are removed. More specifically, the articulating component 30 is formed of hydrogel that consists of a three-dimensional matrix having a molecular structure made of complex polymers. The elastic articulating component 30 is hydrophilic in one aspect. Specifically, the elastic articulating component 30 includes a relatively high proportion of oxygen atoms (i.e. in hydroxy groups), nitrogen atoms, or other non-carbon atoms to provide “polar” groups that attract water.

A sidewall 25 of the anchor body can be formed as a multi-layer sidewall, and include an outer layer 25a that is substantially or generally porous to promote bone ingrowth, and an inner layer 25b that is non-porous and configured to provide a barrier for the elastic articulating component 30. One of ordinary skill in the art would understand that various structure or profiles can be provided on an outer surface or outer layer of the sidewall 25 to promote bone ingrowth once the implant 10 is installed within a recipient site of a patient.

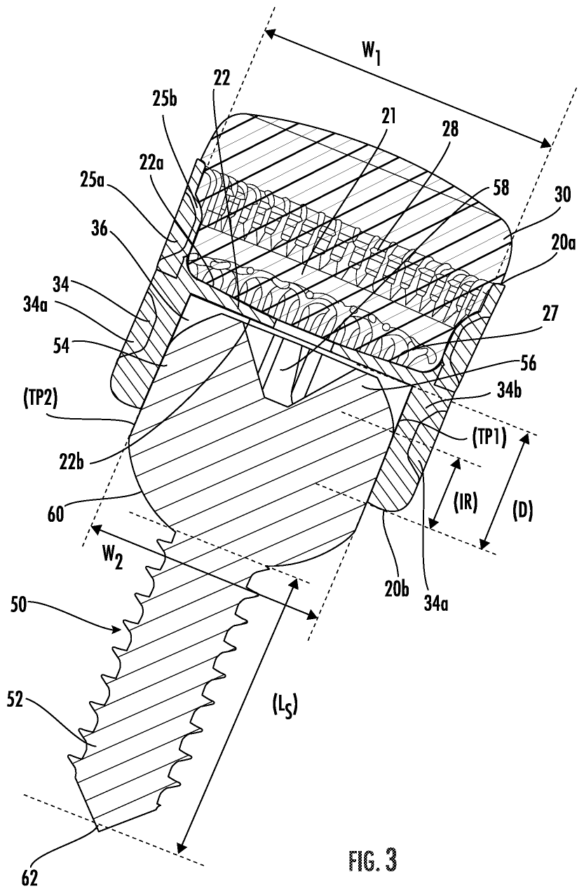

The anchor body 20 of the implant 10 can include an elongated base portion or collar 34 defining a receptacle 36. In one aspect, the receptacle 36 has a completely smooth inner surface. The collar 34 can be defined separately from the sidewall 25 in one aspect, or can be defined integrally with the sidewall 25 in another aspect. As shown in FIG. 3, an outer surface or layer 34a of the collar 34 can be substantially or generally porous to promote bone ingrowth, while an inner layer 34b of the collar can be non-porous and solid. In one aspect, an outer layer or surface of both the sidewall 25 and the collar 34 can have the same porosity or texture, and can be configured to promote bone ingrowth. In one aspect, the sidewall 25 and the collar 34 are formed integrally with each other and generally have the same characteristics, shape, profile, and composition.

The collar 34 can be formed as a sleeve extending downward or axially away from the base 22 of the anchor body 20, which partially defines the cavity 21 for retaining the elastic articulating component 30. The collar 34 can comprise an annular wall portion surrounding the receptacle 36. The collar 34 can be formed integrally with a remainder of the anchor body 20. In one aspect, a radially outer surface of the collar 34 has an identical outer diameter to an outer diameter of the anchor body 20 such that a continuous radially outer surface is defined by the implant 10.

In one aspect, an axial height of the collar 34 is between 75%-150% of an axial height of the sidewall 25. The axial height of the collar 34 corresponds to a depth (D) of the receptacle 36 and is specifically designed to provide sufficient contact surface with a fixation element 50, as described in more detail herein. As shown in FIG. 1B, a bottom rim 38 of the collar 34 can include a chamfered portion or profile. The bottom rim 38 can also include a porous outer surface that is configured to promote bone ingrowth.

The receptacle 36 defined by the collar 34 can have a tapered profile (TP1), in one aspect, which is described in more detail below. In one aspect, the receptacle 36 defines a female Morse taper profile.

FIG. 1C illustrates a fixation element 50. In one aspect, the fixation element 50 is a screw including a threaded portion 52 and a head portion 54. The fixation element 50 could include other types of fastening structures or fixation structures besides a screw threading. The threaded portion 52 can include a tapered profile in one aspect. In another aspect, the threaded portion 52 can have a generally constant profile or outer diameter between the head portion 54 and a tip 62.

The tip 62 is configured to engage with a patient's anatomy and fix the fixation element 50 in place relative to a patient. For example, the tip 62 may be configured to engage with a bone of a patient. In one aspect, the radially outer surface of the head portion 54 is smooth and circular, and may be formed as an annular wall. According to one embodiment, the head portion 54 includes a radially outer surface having a tapered profile (TP2). The taper may run from a larger diameter area adjacent the head portion 54, and a smaller diameter area adjacent the tip 62. In one aspect, the head portion 54 defines a male Morse taper profile. In one aspect, the angle of the taper for each of the tapered profiles (TP1, TP2) is 1.3 degrees to 1.6 degrees. In one aspect, the angle of the taper for each of the tapered profiles (TP1, TP2) is 1.49 degrees (+/− manufacturing tolerances). One of ordinary skill in the art would understand that other types of machine tapers, besides a Morse taper could be used.

The head portion 54 of the fixation element 50 is configured to mate with the receptacle of the collar 34 such that the fixation element 50 engages and is fixedly attached to the anchor body 20. The head portion 54 can include a distal portion 56 that defines a drive interface 58 which is configured to be engaged by a tool, and a proximal portion 60 positioned adjacent to the threaded portion 52. In one aspect, the proximal portion 60 has a curved outer surface profile that provides a transitional area connecting the head portion 54 to the threaded portion 52, as shown in FIG. 3. The distal portion 56 can also include a curved or sloping outer surface profile extending between the head portion 54 and the drive interface 58. In an assembled state, the distal portion 56 of the fixation element 50 is adjacent to or abuts a base 22 of the anchor body 20.

FIG. 3 illustrates an interface region (IR) illustrating an overlap between the tapered profiles (TP1, TP2) of the anchor body 20 and the fixation element 50. As shown in FIG. 3, in one aspect, the interface region (IR) is shorter than an axial length (Ls) of the threaded portion 52. In one aspect, the interface region (IR) is less than 20% of a total axial length (Ls) of the threaded portion 52. In one aspect, the interface region (IR) is at least 50% of an axial depth (D) of the receptacle 36. As shown in FIG. 3, a width (W1) of the cavity 21 can be larger than a width (W2) of the receptacle 36. One of ordinary skill in the art would recognize that the widths (W1, W2) may vary.

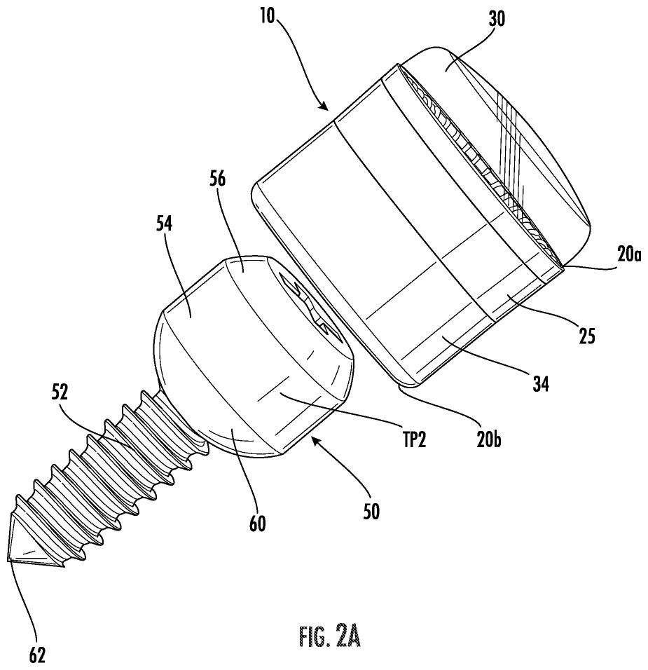

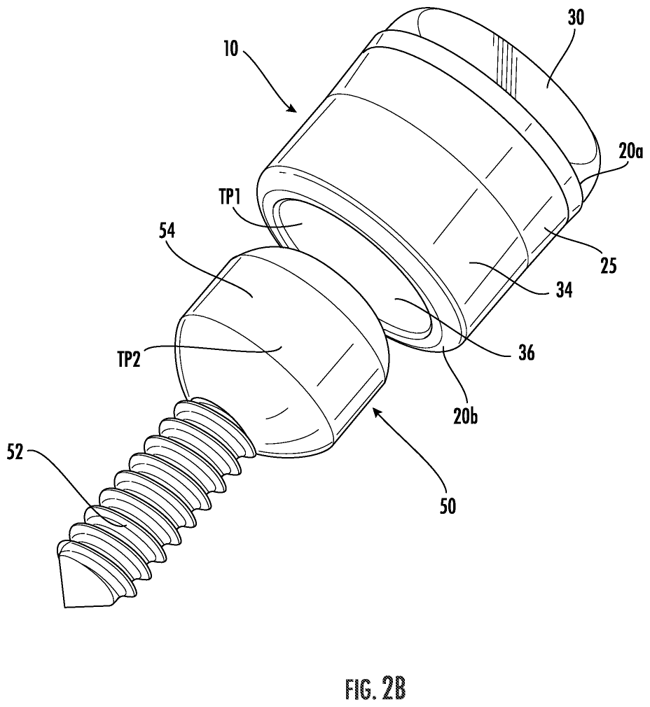

In one aspect, a weld, such as a cold weld, is provided between the fixation element 50 and the anchor body 20. In other words, there is no requirement for any heating, molding, fusion, adhesives, glue, or other attachment elements or procedures to join the fixation element 50 with the anchor body 20 other than impacting the anchor body 20 directly onto the head portion 54. This provides convenience for a surgeon, for example, who can manually perform this attachment process prior to or during surgery, and can attach the components with minimal effort and no additional fastening elements, such as adhesive. Attachment between the fixation element 50 and the anchor body 20 can occur solely and directly due to the interface between the head portion 54 and the receptacle 36. FIGS. 2A and 2B illustrate the fixation element 50 and the implant 10 prior to attachment or insertion.

In one aspect, the base 22 of the anchor body 20 partially defines the cavity 21 on a first surface 22a and defines an abutment surface for a head portion 54 of the fixation element 50 on a second, opposite surface 22b. In one aspect, the head portion 54 is stopped short of abutment with the second surface 22b of the base 22 and instead the second surface 22b directly faces the head portion 54.

In one aspect, multiple fixation elements 50 of varying sizes and dimensions can be provided for a single implant. This allows surgeons to conveniently select a specific fixation element having a particularly depth and thickness of the threaded portion that is most appropriate for a particular patient.

During implantation, the fixation element 50 is secured to a recipient patient site by turning the fixation element 50. The implant 10 is then impacted or pressed onto the head portion 54 of the fixation element 50 to create a Morse taper cold metal bond between the two components. Alternatively, the fixation element 50 can be joined or fastened to the implant 10 first, and then the combination of the implant 10 and the fixation element 50 can be rotated to engage with a patient's anatomy.

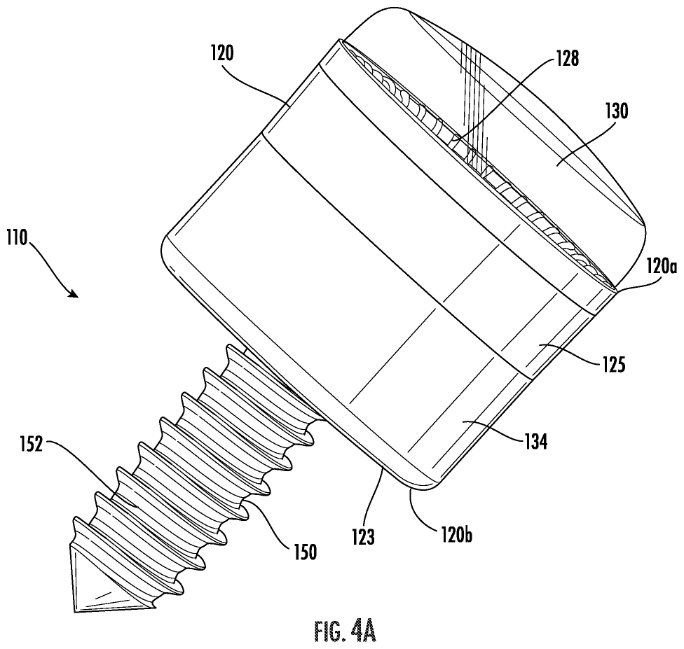

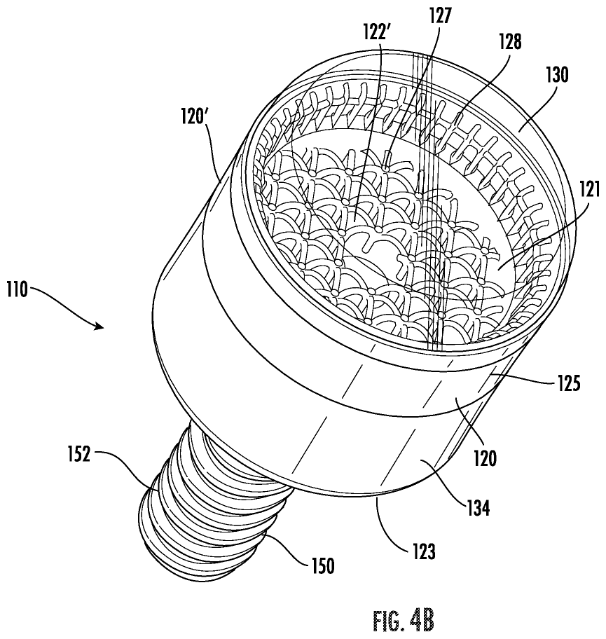

In another aspect shown in FIGS. 4A and 4B, a separately formed fixation element is not required, and instead a fixation element 150 including a threaded portion 152 can be formed directly and integrally with the anchor body 120. Similar to implant 10, the implant 110 also includes a first end 120a of the anchor body 120 that defines the cavity 121 having a base 122′ for retaining the elastic articulating component 130 via engagement with attachment elements 127, 128. A base portion 134 can extend downward from the sidewall 125. An outer surface or layer of the base portion 134 and the sidewall 125 can include a porous structure configured to promote bone ingrowth. As shown in FIGS. 4A and 4B, the threaded portion 152 extends directly from a bottom end 123 of the base portion 134. In this embodiment, the entire implant 110 can be turned such that the threaded portion 152 is screwed into the recipient site. All other aspects of implant 110 are similar to implant 10 unless specified herein.

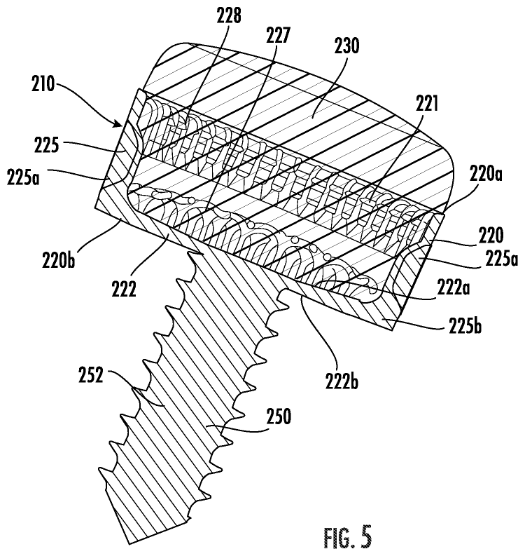

FIG. 5 illustrates another aspect in which the fixation element 250 is integrally formed with the anchor body 220. As shown in FIG. 5, a threaded portion 252 can be formed directly with a base 222 of the implant 210. The implant 210 similarly includes the elastic articulating component 230 within the cavity 221, sidewall 225, attachment elements 227, 228, etc. The sidewall 225 can include a porous outer layer 225a and a non-porous inner layer 225b. The elastic articulating component 230 extends upwards from the first end 220a of the anchor body 220. A fixation element 250 including the threaded portion 252 extends away from the base 222 of the implant 210. This embodiment functions in the same way as described with respect to the implant 110.

A surgeon could rotate or otherwise engage the implant 210 such that the threaded portion 252 engages with the patient's anatomy. In one aspect, the base 222 partially defines the cavity 221 on a first side surface 222a and the base 222 defines an attachment surface for the fixation element 250 on a second, opposite side surface 222b.

Having thus described the presently preferred embodiments in detail, it is to be appreciated and will be apparent to those skilled in the art that many physical changes, only a few of which are exemplified in the detailed description, could be made without altering the inventive concepts and principles embodied therein. It is also to be appreciated that numerous embodiments incorporating only part of the preferred embodiment are possible which do not alter, with respect to those parts, the inventive concepts and principles embodied therein.

The present embodiments and optional configurations are therefore to be considered in all respects as exemplary and/or illustrative and not restrictive, the scope of the invention being indicated by the appended claims rather than by the foregoing description, and all alternate embodiments and changes to this embodiment which come within the meaning and range of equivalency of said claims are therefore to be embraced therein.

What is claimed is:

1. A device configured for use as a medical implant, the device comprising:
an anchor body comprising a first end defining a cavity, and an elastic articulating component retained within the cavity, and a second end including a fixation element extending cantilevered from the anchor body.

2. The device according to claim 1, wherein the second end of the anchor body defines a receptacle, the fixation element is secured within the receptacle, and the fixation element includes a threaded portion that is configured to retain the implant with a patient recipient site.

3. The device according to claim 2, wherein a first width (W1) of the cavity is larger than a second width (W2) of the receptacle.

4. The device according to claim 2, wherein the fixation element is formed separately from the anchor body, and the fixation element further comprises a head portion dimensioned to be retained within the receptacle, and the head portion includes a drive interface configured to be engaged by a tool.

5. The device according to claim 4, wherein the anchor body is configured to be impacted in a downward direction to secure the anchor body onto the head portion, and the head portion engages against a base of the anchor body in an installed state.

6. The device according to claim 1, wherein the fixation element is formed integrally with the anchor body.

7. The device according to claim 1, wherein the anchor body comprises a receptacle having a first tapered profile that is a female Morse taper, and the fixation element comprises a head portion having a second tapered profile that is a male Morse taper, and the female Morse taper of the receptacle and the male Morse taper of the head portion are configured to secure the anchor body with the fixation element via a cold weld or bond.

8. The device according to claim 7, wherein an interface region (IR) is defined between the first and second tapered profiles, and the interface region (IR) is shorter than an axial length (Ls) of a threaded portion of the fixation element.

9. The device according to claim 8, wherein the interface region (IR) is at least 50% of an axial depth of the receptacle.

10. The device according to claim 1, wherein the anchor body further comprises a sidewall partially defining the cavity, and a collar defining a receptacle, wherein the sidewall and the collar each have an outer surface layer having a porosity configured to promote bone ingrowth.

11. The device according to claim 1, wherein the anchor body defines a base that partially defines the cavity on a first surface and defines an attachment surface for the fixation element on a second, opposite surface.

12. The device according to claim 1, wherein the anchor body defines a base that partially defines the cavity on a first surface and defines an abutment surface for a head portion of the fixation element on a second, opposite surface.

13. A device configured for use as a medical implant, the device comprising:
an anchor body including:
a first end having a sidewall partially defining a cavity, and at least one attachment element configured to retain an elastic articulating component, and
a second end comprising a collar defining a receptacle having a first tapered profile that is configured to receive a portion of a fixation element; and
a fixation element including a head portion and a threaded portion, wherein the head portion has a second tapered profile, and the head portion is configured to be inserted and retained within the receptacle via engagement between the first and second tapered profiles, and the threaded portion is configured to retain the device with a patient recipient site.

14. The device according to claim 13, wherein the anchor body includes a base that partially defines the cavity on a first surface, and the base defines a second, opposite surface directly facing the head portion of the fixation element.

15. The device according to claim 13, wherein the base includes the at least one attachment element configured to retain the elastic articulating component.

16. The device according to claim 13, wherein a first width (W1) of the cavity is larger than a second width (W2) of the receptacle.

17. The device according to claim 13, wherein the sidewall and the collar both include an outer layer having a porosity configured to promote bone ingrowth.

18. A device configured for use as a medical implant, the device comprising:
an anchor body including:
a first end having a sidewall partially defining a cavity, and at least one attachment element configured to retain an elastic articulating component, and
a second end comprising a collar that is integrally formed with the sidewall and defines a receptacle having a first tapered profile that is configured to receive a portion of a fixation element; and
a fixation element including a head portion and a threaded portion, wherein the head portion has a second tapered profile, and the head portion is configured to be inserted and retained within the receptacle via engagement between the first and second tapered profiles, and the threaded portion is configured to retain the device with a patient recipient site.

19. The device according to claim 18, wherein the sidewall and the collar have a constant outer diameter, and both include an outer layer having a porosity configured to promote bone ingrowth.

20. The device according to claim 18, wherein the anchor body includes a base that partially defines the cavity on a first surface, and the base defines a second, opposite surface directly facing the head portion of the fixation element.
